(19) United States
(12) Patent Application Publication
(10) Pub. No.: US 2015/0267242 A1
(43) Pub. Date: Sep. 24, 2015

(54) ACYL-COA DEHYDROGENASE ASSAYS

(71) Applicant: ADVANCED LIQUID LOGIC, INC, Research Triangle Park, NC (US)

(72) Inventors: Nora J. Foegeding, Raleigh, NC (US); Carrie A. Graham, Raleigh, NC (US); Allen E. Eckhardt, San Diego, CA (US)

(73) Assignee: ADVANCED LIQUID LOGIC, INC., San Diego, CA (US)

(21) Appl. No.: 14/439,255

(22) PCT Filed: Oct. 30, 2013
(86) PCT No.: PCT/US2013/067390

§ 371 Date: Apr. 29, 2015

Related U.S. Application Data
(60) Provisional application No. 61/722,534, filed on Nov. 5, 2012.

(51) Int. Cl. C12Q 1/32

(52) U.S. Cl. CPC C12Q 1/32; G01N 2333/90206; G01N 2800/04

(57) ABSTRACT

A method for conducting medium-chain acyl-CoA dehydrogenase (MCAD) and very-long-chain acyl-CoA dehydrogenase (VCAD) enzymatic activity assays is provided. The method may include, but is not limited to, preparing a sample; preparing an enzyme-specific substrate/reagent mixture; mixing an aliquot of the prepared sample with an aliquot of the enzyme-specific substrate/reagent mixture; reading absorbance in the range of about 600 nm; incubating the prepared sample and enzyme-specific substrate/reagent mixture; and reading absorbance in the range of about 600 nm at various time intervals.

### 1 RELATED APPLICATIONS

In addition to the patent applications cited herein, each of which is incorporated herein by reference, this patent application is related to and claims priority to U.S. Provisional Patent Application No. 61/722,534, filed on Nov. 5, 2012, entitled “Acyl-CoA Dehydrogenase Assays”, the entire disclosures of which is incorporated herein by reference.

### 2 FIELD OF THE PRESENT DISCLOSURE

The present disclosure relates to methods for enzymatic detection of medium-chain acyl-CoA dehydrogenase and very-long-chain acyl-CoA dehydrogenase deficiency.

### 3 BACKGROUND

Medium-chain acyl-CoA dehydrogenase deficiency (MCADD) and very-long-chain acyl-CoA dehydrogenase deficiency (VLCADD) are two inborn errors of metabolism that are part of the fatty acid oxidation group of disorders. Fatty acid oxidation is essential during prolonged fasting and/or periods of increased energy demands, when energy production relies increasingly on fat metabolism. If there is an abnormality in fatty acid metabolism, then life-threatening episodes of metabolic decompensation may ensue. For newborn infants, early detection (e.g., within the first few hours of life) is essential for establishing a diagnosis before the onset of symptoms. Fatty acid oxidation disorders are included in many newborn screening (NBS) programs that are typically performed at centralized laboratories. However, the turnaround time for reporting NBS results is often relatively long (e.g., about 3 days). Therefore, there is a need for rapid, point-of-care testing methods for fatty oxidation disorders (e.g., MCADD, VLCADD).

### 4 BRIEF DESCRIPTION OF THE PRESENT DISCLOSURE

In one embodiment, the present disclosure provides a method for conducting medium-chain acyl-CoA dehydrogenase (MCAD) and very-long-chain acyl-CoA dehydrogenase (VCAD) enzymatic activity assays. The method may include, but is not limited to, preparing a sample; preparing an enzyme-specific substrate/reagent mixture; mixing an aliquot of the prepared sample with an aliquot of the enzyme-specific substrate/reagent mixture; reading absorbance in the range of about 600 nm; incubating the prepared sample and enzyme-specific substrate/reagent mixture; and reading absorbance in the range of about 600 nm at various time intervals. The sample may include a blood sample. The enzymatic activity assays may be conducted in one of fresh or fresh-frozen whole blood samples. The enzymatic activity assays may be conducted in dried blood spot (DBS) extracts. The enzyme-specific substrate for MCAD may include octanoyl-Coenzyme A. The enzyme-specific substrate for VLCAD may include palmitoyl-Coenzyme A. The enzymatic activity may be measured by reduction of 2,6-dichlorophenolindophenol (DCPIP) at 600 nm. The reaction scheme for reduction of DCPIP may include:

Substrate-CoA+sample+DCPIP_{oxidized}→Substrate-CoA_{oxidized}+DCPIP_{reduced}

The whole blood sample may be prepared using a freeze/thaw lysis protocol to lyse red blood cells. The sample may be diluted prior to assaying. The sample may be diluted with a quantity of extraction buffer. The extraction buffer may include in the range of about 0.1% (w/v) Tween® 20 in molecular grade water. The DBS extracts may be prepared from blood samples collected and dried on filter paper. The extraction of the DBS extract may include, adding an aliquot of extraction buffer to the blood sample dried on filter paper; and incubating in the range of about 30 minutes in the range of about 1600 rpm on a plate shaker at about room temperature. The enzymatic activity assays may be conducted on-bench. Conducting the enzymatic activity assays on-bench may include using a multi-well microtiter plate assay and plate reader. The sample may include incubating the sample prior to mixing the sample with the enzyme-specific substrate/ reagent mixture. Preparing the enzyme-specific substrate/reagent mixture may include incubating the enzyme-specific substrate/reagent mixture prior to mixing the enzyme-specific substrate/reagent mixture with the sample. Incubating may be conducted in the range of about 37° C.

### 5 BRIEF DESCRIPTION OF THE DRAWINGS

FIG. 1 shows a bar graph of an example of an MCAD assay performed on-bench using whole blood samples.

### 6 DEFINITIONS

As used herein, the following terms have the meanings indicated.

“Activate,” with reference to one or more electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 1000 V, or about 300 V. Where alternating current is used, any suitable frequency may be employed. For example, an electrode may be activated using alternating current having a frequency from about 1 Hz to about 10 MHz, or from about 10 Hz to about 60 Hz, or from about 20 Hz to about 40 Hz, or about 30 Hz.

“Bubble” means a gaseous bubble in the filler fluid of a droplet actuator. In some cases, bubbles may be intentionally included in a droplet actuator, such as those described in U.S. Patent Pub. No. 20100190263, entitled “Bubble Techniques for a Droplet Actuator,” published on Jul. 29, 2010, the entire disclosure of which is incorporated herein by references. The present present disclosure relates to undesirable bubbles which are formed as a side effect of various processes within a droplet actuator, such as evaporation or hydrolysis of a droplet in a droplet actuator. A bubble may be at least partially bounded by filler fluid. For example, a bubble may be completely surrounded by filler fluid or may be bounded by filler fluid and one or more surfaces of the droplet actuator. As another example, a bubble may be bounded by filler fluid, one or more surfaces of the droplet actuator, and/or one or more droplets in the droplet actuator.

“Droplet” means a volume of liquid on a droplet actuator that is at least partially bounded by a filler fluid. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the present disclosure, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. A droplet can include nucleic acids, such as DNA, genomic DNA, RNA, mRNA or analogs thereof; nucleotides such as deoxyribonucleotides, ribonucleotides or analogs thereof such as analogs having terminator moieties such as those described in Bentley et al., Nature 456:53-59 (2008), WO 04/018497; U.S. Pat. No. 7,057,026; WO 91/06678; WO 07/123744; U.S. Pat. No. 7,329,492; U.S. Pat. No. 7,211,414; U.S. Pat. No. 7,315,019; U.S. Pat. No. 7,405,281, and US 2008/0108082, each of which is incorporated herein by reference; enzymes such as polymerases, ligases, recombinases, or transposases; binding partners such as antibodies, epitopes, streptavidin, avidin, biotin, lectins or carbohydrates; or other biochemically active molecules. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids. A droplet may include one or more beads.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000; Kim and/or Shah et al., U.S. patent application Ser. No. 10/343,261, entitled “Electrowetting-driven Micropumping,” filed on Jan. 27, 2003, Ser. No. 11/275,668, entitled “Method and Apparatus for Promoting the Complete Transfer of Liquid Drops from a Nozzle,” filed on Jan. 23, 2006, Ser. No. 11/460,188, entitled “Small Object Moving on Printed Circuit Board,” filed on Jan. 23, 2006, Ser. No. 12/465,935, entitled “Method for Using Magnetic Particles in Droplet Microfluidics,” filed on May 14, 2009, and Ser. No. 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. No. 7,641,779, entitled “Method and Apparatus for Programmable fluidic Processing,” issued on Jan. 5, 2010, and U.S. Pat. No. 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a droplet operations gap between them and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, the dielectric layers and/or the electrodes forming a droplet operations surface. A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the present disclosure. During droplet operations it is preferred that droplets remain in continuous contact or frequent contact with a ground or reference electrode. A ground or reference electrode may be associated with the top substrate facing the gap, the bottom substrate facing the gap, and/or in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material. Where multiple substrates are used, a spacer may be provided between the substrates to determine the height of the gap therebetween and define dispensing reservoirs. The spacer height may, for example, be at least about 5 μm, 100 μm, 200 μm, 250 μm, 275 μm or more. Alternatively or additionally the spacer height may be at most about 600 μm, 400 μm, 350 μm, 300 μm, or less. The spacer may, for example, be formed of a layer of projections form the top or bottom substrates, and/or a material inserted between the top and bottom substrates. One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be effected by the droplet operations electrodes using the liquid. The base (or bottom) and top substrates may in some cases be formed as one integral component. One or more reference electrodes may be provided on the base (or bottom) and/or top substrates and/or in the gap. Examples of reference electrode arrangements are provided in the above referenced patents and patent applications. In various embodiments, the manipulation of droplets by a droplet actuator may be electrode mediated, e.g., electrowetting mediated or dielectrophoresis mediated or Coulombic force mediated. Examples of other techniques for controlling droplet operations that may be used in the droplet actuators of the present disclosure include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed to conduct a droplet operation in a droplet actuator of the present disclosure. Similarly, one or more of the foregoing may be used to deliver liquid into a droplet operations gap, e.g., from a reservoir in another device or from an external reservoir of the droplet actuator (e.g., a reservoir associated with a droplet actuator substrate and a flow path from the reservoir into the droplet operations gap). Droplet operations surfaces of certain droplet actuators of the present disclosure may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), other fluorinated monomers for plasma-enhanced chemical vapor deposition (PECVD), and organosiloxane (e.g., SiOC) for PECVD. In some cases, the droplet operations surface may include a hydrophobic coating having a thickness ranging from about 10 nm to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness of at least about 20 nm, 50 nm, 75 nm, 100 nm or more. Alternatively or additionally the thickness can be at most about 200 nm, 150 nm, 125 nm or less. I In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.); NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass), PARYLENE™ N, and PARYLENE™ HT (for high temperature, ˜300° C.) (available from Parylene Coating Services, Inc., Katy, Tex.); TEFLON® AF coatings; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; and polypropylene. Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the present disclosure may be derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, other fluorinated monomers for plasma-enhanced chemical vapor deposition (PECVD), and organosiloxane (e.g., SiOC) for PECVD. Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan. Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the methods and apparatus described herein includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance or capacitance sensing or imaging techniques may sometimes be used to determine or confirm the outcome of a droplet operation. Examples of such techniques are described in Sturmer et al., International Patent Pub. No. WO/2008/101194, entitled “Capacitance Detection in a Droplet Actuator,” published on Aug. 21, 2008, the entire disclosure of which is incorporated herein by reference. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may exceed about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to electrowetting area; in other words, 1×-, 2×- 3×-droplets are usefully controlled operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid, such as a gas or liquid, associated with a droplet operations substrate of a droplet actuator, which fluid is sufficiently immiscible with a droplet phase to render the droplet phase subject to electrode-mediated droplet operations. For example, the droplet operations gap of a droplet actuator is typically filled with a filler fluid. The filler fluid may, for example, be a low-viscosity oil, such as silicone oil or hexadecane filler fluid. The filler fluid may fill the entire gap of the droplet actuator or may coat one or more surfaces of the droplet actuator. Filler fluids may be conductive or non-conductive. Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the present disclosure are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein.

“Reservoir” means an enclosure or partial enclosure configured for holding, storing, or supplying liquid. A droplet actuator system of the present disclosure may include on-cartridge reservoirs and/or off-cartridge reservoirs. On-cartridge reservoirs may be (1) on-actuator reservoirs, which are reservoirs in the droplet operations gap or on the droplet operations surface; (2) off-actuator reservoirs, which are reservoirs on the droplet actuator cartridge, but outside the droplet operations gap, and not in contact with the droplet operations surface; or (3) hybrid reservoirs which have on-actuator regions and off-actuator regions. An example of an off-actuator reservoir is a reservoir in the top substrate. An off-actuator reservoir is typically in fluid communication with an opening or flow path arranged for flowing liquid from the off-actuator reservoir into the droplet operations gap, such as into an on-actuator reservoir. An off-cartridge reservoir may be a reservoir that is not part of the droplet actuator cartridge at all, but which flows liquid to some portion of the droplet actuator cartridge. For example, an off-cartridge reservoir may be part of a system or docking station to which the droplet actuator cartridge is coupled during operation. Similarly, an off-cartridge reservoir may be a reagent storage container or syringe which is used to force fluid into an on-cartridge reservoir or into a droplet operations gap. A system using an off-cartridge reservoir will typically include a fluid passage means whereby liquid may be transferred from the off-cartridge reservoir into an on-cartridge reservoir or into a droplet operations gap.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface. In one example, filler fluid can be considered as a film between such liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 7 DESCRIPTION

The present invention provides methods for enzymatic detection of medium-chain acyl-CoA dehydrogenase and very-long-chain acyl-CoA dehydrogenase deficiency. In various embodiments, the invention provides methods for conducting MCAD and VLCAD enzymatic activity assays in fresh blood samples, fresh-frozen blood samples, and dried blood spot (DBS) samples. In one example, the enzymatic assays for MCAD and VLCAD activities are performed on-bench using a 96-well microtiter plate format. In another example, the enzymatic assays for MCAD and VLCAD activities are performed in droplets in oil. The droplet-based method includes, among other things, incubating a droplet in oil, the droplet comprising a substrate liquid and a sample liquid.

In one example, the enzymatic assays for MCAD and VLCAD are used for newborn testing for MCAD deficiency (MCADD) and VLCAD deficiency (VLCADD), respectively. A deficiency in both MCAD and VLCADD enzyme activities is indicative of multiple acyl-CoA dehydrogenase deficiency (MADD).

7.1 MCAD and VLCAD Enzymatic Assays

Acyl-CoA dehydrogenases (e.g., MCAD or VLCAD) are a class of chain-length specific enzymes that function to catalyze the initial step in each cycle of fatty acid β-oxidation in the mitochondria of cells. The methods of the invention use a common assay format for the detection of MCAD and VLCAD activities. The assay format uses enzyme-specific substrates and a common electron acceptor cascade for colorimetric detection of MCAD or VLCAD activities. The detection chemistry is a colorimetric assay based on the reduction of 2,6-dichlorophenolindophenol (DCPIP) and the oxidation of an enzyme-specific substrate in the presence of phenzaine methosulfate (PMS) as an intermediate electron acceptor. The enzyme-specific substrate for MCAD is octanoyl-Coenzyme A. The enzyme-specific substrate for VLCAD is palmitoyl-Coenzyme A. Enzymatic activity is measured by reduction of DCPIP at 600 nm. The reaction scheme is as follows:

Substrate-CoA+sample+DCPIP_{oxidized}→Substrate-CoA_{oxidized}+DCPIP_{reduced}

Inhibitors, such as spiropentaneacetic acid (SPA), which is specific for MCAD, may be used to show assay specificity.

In one example, the enzymatic assays of the invention are performed in fresh or fresh-frozen whole blood samples. For example, the whole blood sample may be lysed using a freeze/thaw protocol and analyzed directly. Alternatively, the lysed whole blood sample may be stored at −80° C. until use. The blood samples are diluted prior to analysis. For example, a 10 μL aliquot of lysed whole blood is diluted with 320 μL of extraction buffer (e.g., 0.1% (w/v) Tween® 20 in molecular grade water).

In another example, the enzymatic assays of the invention may be performed in dried blood spot (DBS) extracts. DBS extracts may, for example, be prepared from blood samples collected and dried on filter paper. A manual or automatic puncher may be used to punch a sample, e.g., a 3 mm-punch. Each punch may be placed into a separate well of a round bottomed 96-well plate. An aliquot (e.g., 100 μL) of extraction buffer such as 0.1% (w/v) Tween® 20 in molecular grade water may be added to each well that contains a DBS punch and incubated for about 30 minutes at 1600 rpm on a plate shaker at room temperature to extract the DBS samples. Extraction buffer composition (e.g., pH, detergent concentration, salts, etc.) may be selected for performance with reagents used in specific assay protocols.

In one example, the methods of the invention include, but are not limited to, the following steps:
1. Preparing a sample, e.g. a blood sample;
2. Preparing an enzyme-specific substrate formulation;
3. Preparing a enzyme-specific substrate/reagent mixture;
4. Mixing an aliquot of prepared sample (e.g., blood sample) with an aliquot of substrate/reagent mixture;
5. Reading absorbance at 600 nm (t=0);
6. Incubating the sample at about 37° C.; and
7. Reading absorbance at 600 nm at different time intervals (e.g., t=10 min, t=30 min, t=120 min, and t=240 min)

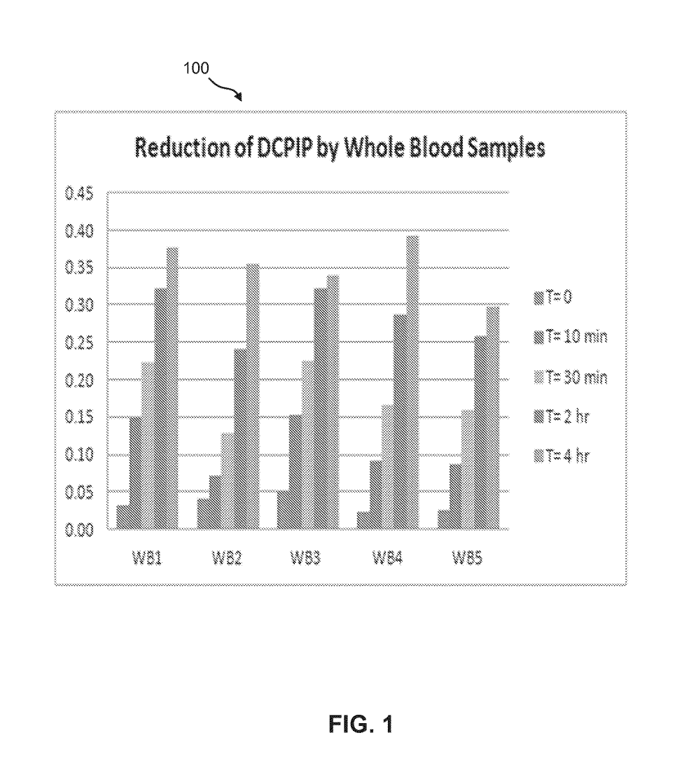

FIG. 1 shows a bar graph of an example of a MCAD assay performed on-bench using whole blood samples. The substrate formulation was 1.0 mM octanoyl-CoA in 100 mM sodium phosphate pH 7.0. Five whole blood samples (i.e., WB1 through WB5) were prepared using a freeze/thaw lysis protocol to lyse red blood cells and stored at −80° C. until use. Dilutions of each lysed whole blood sample were prepared by diluting 10 μL of lysed whole blood into 320 μL of 0.1% w/v Tween-20 in molecular grade water. The assay was performed on-bench using a 96-well microtiter plate assay and plate reader.

The assay protocol was conducted as follows. For each whole blood sample, a reagent mixture comprising 50 μL of 600 μM DCPIP, 50 μL of 400 μM PMS, and 50 μL of 1.0 mM octanoyl-CoA was prepared and placed in a separate holding well of a 96-well microtiter plate. Aliquots (50 μL) of each diluted whole blood sample (i.e., WB1 through WB5) were placed in corresponding separate wells of the 96-well microtiter plate. The microtiter plate was covered and incubated at 37° C. for 40 minutes. After the incubation period, the reagent mixtures in the holding wells were transferred to the corresponding diluted whole blood sample wells. Absorbance was immediately read at 600 nm (t=0). The plate was covered again and incubated at 37° C. Absorbance readings at 600 nm were obtained at t=10 min, t=30 min, t=120 min, and t=240 min. Control assays for each sample and time point were performed in parallel. For the control assays, the MCAD-specific octanoyl-CoA substrate was replaced with 50 μL of 100 mM sodium phosphate pH 7.0. The data is expressed as sample absorbance minus control absorbance. The data shows the change in absorbance of the five different lysed whole blood samples, corresponding to MCAD activity over time.

The 96-well microtiter plate assay may be translated into a droplet-based format and microfluidic volumes. For example, integer mixing ratios are preferred for digital microfluidics which may necessitate adjustment of reagent stock concentrations.

### 8 CONCLUDING REMARKS

The foregoing detailed description of embodiments refers to the accompanying drawings, which illustrate specific embodiments of the present disclosure. Other embodiments having different structures and operations do not depart from the scope of the present disclosure. The term “the present disclosure” or the like is used with reference to certain specific examples of the many alternative aspects or embodiments of the applicants' present disclosure set forth in this specification, and neither its use nor its absence is intended to limit the scope of the applicants' present disclosure or the scope of the claims. This specification is divided into sections for the convenience of the reader only. Headings should not be construed as limiting of the scope of the present disclosure. The definitions are intended as a part of the description of the present disclosure. It will be understood that various details of the present disclosure may be changed without departing from the scope of the present disclosure. Furthermore, the foregoing description is for the purpose of illustration only, and not for the purpose of limitation.

1. A method for conducting medium-chain acyl-CoA dehydrogenase (MCAD) and very-long-chain acyl-CoA dehydrogenase (VCAD) enzymatic activity assays; the method comprising:
(a) preparing a sample;
(b) preparing an enzyme-specific substrate/reagent mixture;
(c) mixing an aliquot of the prepared sample with an aliquot of the enzyme-specific substrate/reagent mixture;
(d) reading absorbance in the range of about 600 nm;
(e) incubating the prepared sample and enzyme-specific substrate/reagent mixture; and
(f) reading absorbance in the range of about 600 nm at various time intervals.

2. The method of claim 1 wherein the sample comprises a blood sample.

3. The method of claim 1 wherein the enzymatic activity assays are conducted in one of fresh or fresh-frozen whole blood samples.

4. The method of claim 1 wherein the enzymatic activity assays are conducted in dried blood spot (DBS) extracts.

5. The method of claim 1 wherein the enzyme-specific substrate for MCAD comprises octanoyl-Coenzyme A.

6. The method of claim 1 wherein the enzyme-specific substrate for VLCAD comprises palmitoyl-Coenzyme A.

7. The method of claim 1 following wherein the enzymatic activity is measured by reduction of 2,6-dichlorophenolindophenol (DCPIP) at 600 nm.

8. The method of claim 7 wherein the reduction of DCPIP comprises a reaction scheme comprising:
Substrate-CoA+sample+DCPIP_{oxidized}→Substrate-CoA_{oxidized}+DCPIP_{reduce}

9. The method of claim 3 wherein the whole blood sample is prepared using a freeze/thaw lysis protocol to lyse red blood cells.

10. The method of claim 1 wherein the sample is diluted prior to assaying.

11. The method of claim 10 wherein the sample is diluted with a quantity of extraction buffer.

12. The method of claim 11 wherein the extraction buffer comprises in the range of about 0.1% (w/v) Tween® 20 in molecular grade water.

13. The method of claim 4 wherein DBS extracts are prepared from blood samples collected and dried on filter paper.

14. The method of claim 13 wherein extraction of the DBS extract comprises, adding an aliquot of extraction buffer to the blood sample dried on filter paper; and incubating in the range of about 30 minutes in the range of about 1600 rpm on a plate shaker at about room temperature.

15. The method of claim 1 wherein the enzymatic activity assays are conducted on-bench.

16. The method of claim 15 wherein conducting the enzymatic activity assays on-bench comprises using a multi-well microtiter plate assay and plate reader.

17. The method of claim 1 wherein preparing the sample comprises incubating the sample prior to mixing the sample with the enzyme-specific substrate/reagent mixture.

18. The method of claim 1 wherein preparing the enzyme-specific substrate/reagent mixture comprises incubating the enzyme-specific substrate/reagent mixture prior to mixing the enzyme-specific substrate/reagent mixture with the sample.

19. The method of claim 1 wherein incubating is conducted in the range of about 37° C.

20. The method of claim 1 wherein after incubating the prepared sample and enzyme-specific substrate/reagent mixture the absorbance is read absorbance in the range of about 600 nm at time intervals of about at 10 minutes, at about 30 minutes, at about 120 minutes, and at about 240 minutes.
